(19) United States
(12) Patent Application Publication
(10) Pub. No.: US 2018/0344300 A1
(43) Pub. Date: Dec. 6, 2018

(54) SELF-COLLECTION DEVICE AND KIT FOR COLLECTING CERVICAL AND VAGINAL CELLS

(71) Applicant: 4WOMEN HEALTH, INC., Chicago, IL (US)

(72) Inventors: Courtney Kagan Burrows, Chicago, IL (US); Camille Rose Emma, Brooklyn, NY (US); Arshiya Zehra Fazal, Chicago, IL (US); Sue Hee Kim, Chicago, IL (US); Sang Chai Park, Chicago, IL (US)

(21) Appl. No.: 15/993,181

(22) Filed: May 30, 2018

Related U.S. Application Data
(60) Provisional application No. 62/513,170, filed on May 31, 2017.

(51) Int. Cl. A61B 10/02; A61B 10/00

(52) U.S. Cl. CPC A61B 10/0291; A61B 10/0096

(57) ABSTRACT

A self-collection device and kit that allow for self-collection of vaginal and cervical cells are provided. The device includes an intra-vaginal insertion member and a shaped collection member for collecting vaginal and cervical cells, resembling a menstrual cup.

### CROSS-REFERENCE TO RELATED APPLICATIONS

The present application claims the benefit of priority of U.S. Provisional Patent Application Ser. No. 62/513,170, filed May 31, 2017, the disclosure of which is incorporated herein by reference in its entirety.

### FIELD OF THE INVENTION

The technology relates to the general field of healthcare, and has certain specific application to cervical cancer screening.

### BACKGROUND OF THE INVENTION

Human papillomavirus (HPV) is the most common sexually transmitted infection. HPV has been shown to cause cancers of the cervix, vagina, and vulva. Cervical cancer is the fourth most common cancer among women worldwide and leads to nearly 300,000 deaths annually. In the US, cervical cancer was once one of the most common cancers, but the introduction of the Pap smear in the 1940s drastically decreased this number. A pap smear is an invasive procedure in which cervical cells are manually scraped from the cervix and examined under a microscope for abnormalities.

Current guidelines recommend women aged 21-29 receive a pap smear every three years, and women over 30 receive a pap smear every three years or both a pap smear and an HPV test every five years. In 2013, the CDC reported that 69.4% of women aged 18 and over in the US had received a pap smear in the past three years. While the Pap smear has succeeded in reducing deaths from cervical cancer, most women find the experience uncomfortable, unpleasant, and even painful. For many women, going to the doctor for a pap smear can be a barrier—either due to inconvenience, access, fear, or embarrassment.

Recent studies have shown that a newer cervical cancer screening method, an HPV DNA test, is more sensitive and hence effective in identifying the presence of HPV. Today, physicians collect cells for the HPV DNA test during the pap exam. There is a need for a better, less invasive way to collect cells and DNA to test for HPV.

### DESCRIPTION OF THE PRIOR ART

Of interest to the present invention are devices that seek to collect vaginal fluid and cells using a tampon-like device or absorbent media. Three such devices are described in U.S. Pat. No. 5,725,481, U.S. Pat. No. 3,850,160, and U.S. Pat. No. 6,936,013. According to these references, devices are provided which include absorbent cores formed from highly absorbent materials, such as rayon, cellulose, cotton, or other natural or synthetic fibers. Such devices are limited in their utility because DNA cannot be easily separated from such fibrous materials. Likewise, prior art is used to collect cell samples for pap smears versus an HPV DNA test.

According to other prior art methods a diagnostic tampon is covered with a film of polycarbonate or porous sheet. After the diagnostic tampon is withdrawn from the vaginal cavity, the film of polycarbonate or porous sheet must be removed from the tampon, using a surgical scalpel, and fixed on a slide for examination. Such a device is limited in its utility because it is not adapted to current lab processes and workflows, and because cells cannot be used for DNA testing.

Accordingly, there remains a need in the art for improved devices, methods and kits for cervical cancer screening.

### BRIEF SUMMARY OF THE INVENTION

The present invention relates to improved self-collection devices, kits and methods for cervical cancer screening. Specifically, the invention provides self-collection devices comprising an intra-vaginal insertion member; a shaped collection member for collecting vaginal and cervical cells and a retrieving member.

The collection member is preferably fabricated from a flexible elastomer with silicone being particularly preferred. The material is selected so cells can be readily removed from the device into a bio-stabilizing solution thus avoiding extra steps such as removing a film from the collection device. According to one aspect of the invention the collection member has a cupular shape sized to conform to the vagina such as in menstrual cups well known in the art. According to a further aspect of the invention the collection member comprises protrusions on its exterior which are disposed to create more surface area to collect cells without compromising the comfort of the user. Such protrusions can be circumferential ridges or raised protuberances of various dimensions and designs. According to a further aspect of the invention the collection member can have a partially cupular design but also comprise a plurality of petals expanding from a central core.

The self-collection devices further comprise an intra-vaginal insertion member similar to applicators used with conventional menstrual tampons. According to one aspect of the invention the intra-vaginal insertion member can comprise a cylindrical member which can enclose the collection member for storage and during insertion into the vagina of the collection member. The insertion member can then be withdrawn leaving the collection member in place for collection of vaginal and cervical cells.

The self-collection devices further comprise a retrieving member for retrieving the collection member which is typically a string or other filament such as typically used with menstrual tampons.

Methods are provided by which the self-collection devices are used to collect vaginal and cervical cells for analysis by which the shaped collection member of the self-collection device is inserted into the vagina of a subject to be tested for a sufficient time to collect a biological sample therefrom. The collection member is then withdrawn and is disposed in a tube containing a bio-stabilizing liquid for storage and transport and the biological sample is then transported to a testing laboratory in which the cells, DNA and other biological materials so collected can be analyzed.

Also provided are kits comprising the self-collection devices described above, a container with bio-stabilizing solution, a storage and transport tube comprising a cap and instructions.

### BRIEF DESCRIPTION OF THE DRAWINGS

Various embodiments of the invention are disclosed in the following detailed description and accompanying drawings:

FIG. 1 depicts a perspective view illustrating the kit, which allows for self-collection of cervical cells for cervical cancer screening;

FIG. 2 depicts a perspective view illustrating the self-collection device, which includes a self-collection device and an inserted portion resembling a menstrual cup;

FIG. 3 depicts a perspective view illustrating different embodiments for the inserted portion of the self-collection device;

FIG. 4 depicts a perspective view illustrating the process steps of using the self-collection device and pieces of the kit;

FIG. 5 depicts a perspective view illustrating the test tube and test tube cap, which includes a flexible pouch for holding and releasing the bio-stabilizing liquid and

FIG. 6 depicts a flowchart illustrating the steps to use the self-collection device and kit.

### DETAILED DESCRIPTION OF THE INVENTION

The following is a detailed description of exemplary embodiments to illustrate the principles of the invention. The scope of the invention encompasses numerous alternatives, modifications and equivalent. Numerous specific details are set forth in the following description in order to provide a thorough understanding of the invention. However, the invention may be practiced according to the claims without some or all of these specific details.

The kit is used for cervical cancer screening. The kit includes a self-collection device, specimen bag, test tube with bio-stabilizing solution, and instructions. The self-collection device resembles a tampon and comprises two components: a tampon applicator and an inserted portion that collects cervical cells and vaginal cells. The tampon applicator is made of medical grade plastic, and the inserted portion is made of medical grade, flexible silicone for easy sterilization, comfort, and conformity to the vagina. Both materials are commonly used by women, considered safe, and FDA approved.

The inserted portion is used to collect cervical cells and resembles a menstrual cup; it has ridges, bumps, extensions, and/or brush-like texture to increase surface area and friction for cell collection. The inserted portion is inside the tampon applicator. Similar to the experience of using a tampon, the user will use the tampon applicator to insert the inserted portion into the vagina. After the inserted portion is inside the vagina and ready for removal, the user will remove the inserted portion using the attached string. This is similar to how a tampon is removed from the vagina.

After the inserted portion of the device is removed, a woman will drop it into a test tube, and then screws on a lid which contains a bio-stabilizing liquid. Once the tube is sealed, the woman will push down a button on top of the lid to release the bio-stabilizing liquid into the tube. This bio-stabilizing liquid preserves the sample at room temperature and deactivates any live pathogens to allow for safe and stable shipping. The bio-stabilizing liquid can be methanol-based and will preserve DNA. The kit can use any bio-stabilizing liquid used by physicians and or clinicians to stabilize cells for pap smears with the methanol based solution available commercially as ThinPrep PreservCyt Solution (Hologic®) being particularly preferred.

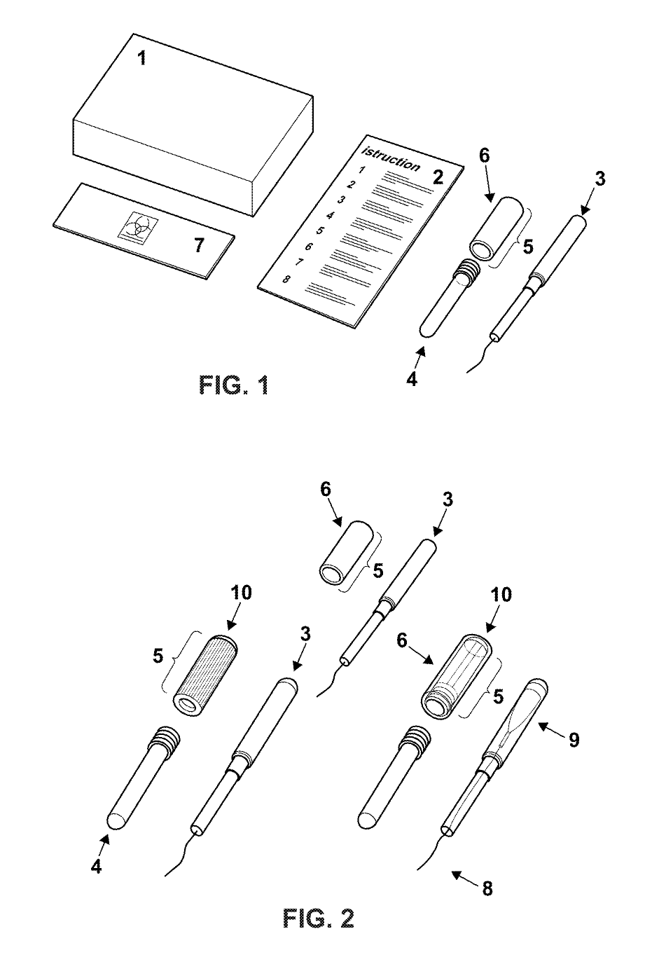

FIG. 1 illustrates the components of the HPV testing kit which includes a box (1), instructions (2), a self-collection device (3), test tube (4), test tube cap (5) holding bio-stabilizing solution (6), and a specimen bag (7).

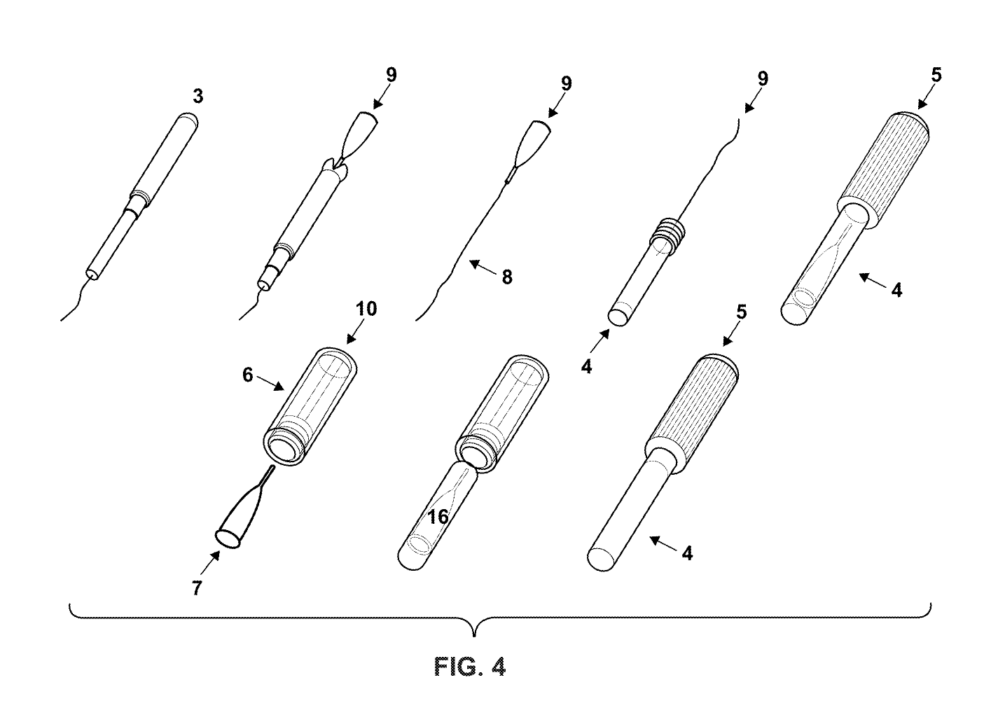

Each component of the kit has a specific purpose. The box (1) holds all of the components and is sent to the user. Instructions (2) provide visual and written instructions for a user. These instructions will outline the steps a user would take as described below with reference to FIG. 4. The intra-vaginal insertion member (3) is inserted into the vagina for collection of cervical and vaginal cells. As shown in more detail in FIG. 2, the self-collection device includes an intra-vaginal insertion member (3) and an inserted portion (9), which is a silicone cup resembling a mini menstrual cup, is disposed inside as depicted. The test tube (4) holds this inserted portion of the device after it is used. The test tube cap (5) holds and releases methanol based bio-stabilizing solution (6) which is used to maintain the DNA. The specimen collection bag (7) is used to hold the test tube (4) and test tube cap (5) for shipping. The test tube (4) and cap (5) is placed inside the specimen bag (7) and mailed to a lab for processing.

FIG. 2 shows the self-collection device and test tube in more detail. Identical to FIG. 1, the intra-vaginal insertion member (3), test tube (4), and test tube cap (5) are pictured. Additionally, the self-collection device 3 contains a shaped collection member (9) with attached string (8) at the bottom end and a button (10) for dispensing the bio-stabilizing solution. The shaped collection member (9) is about 1.5 to 2 inches long and 0.5 to 1 inches wide. These measurements are approximate and may change. It will be made of medical grade, flexible silicone for easy sterilization, comfort, and conformity to the vagina. The purpose of using silicone is that the material can collect cells when inserted into the vagina by rubbing against the vaginal canal. Likewise, the material allows cells to drop off the device once it is in bio-stabilizing liquid. The silicone is also flexible, allowing the inserted portion to conform to the user's vagina and increase comfort. A string (8) attached to the menstrual cup allows for easy removal of the device, and a removal process familiar to women who currently use tampons.

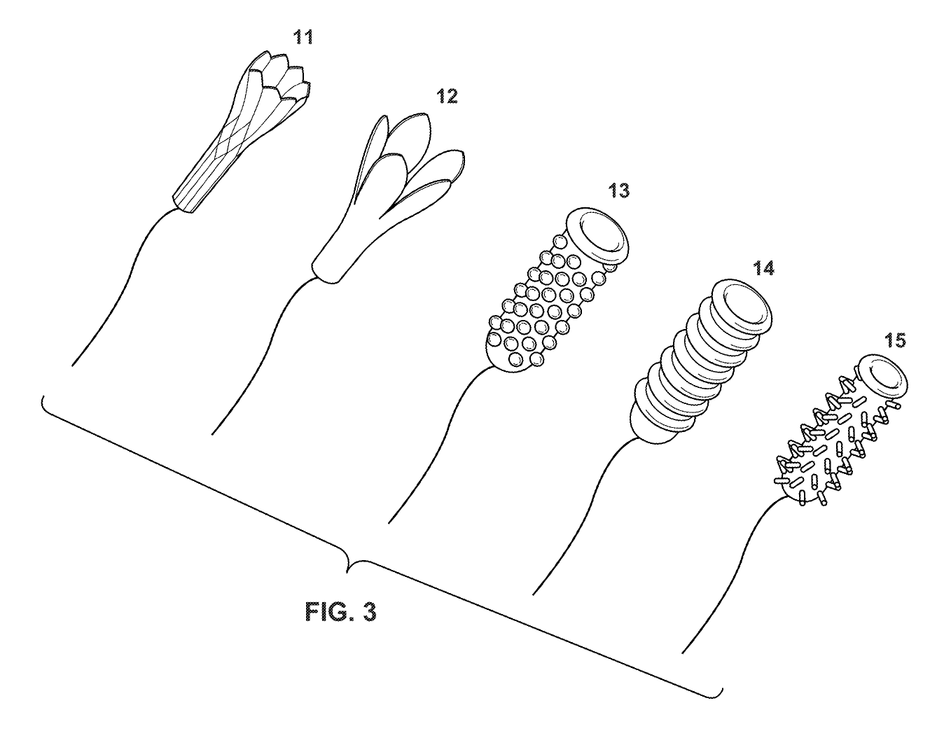

FIG. 3 represents the different embodiments of the inserted portion of the self-collection device (9). It is a mini silicone cup but can take on several embodiments, including different shapes and textures. The embodiments are provided to illustrate aspects of the invention, but the invention is not limited to any embodiment. A flower like structure comprising a plurality of petals expanding from a central core (11) and (12), FIG. 3, allows for a wider rim and a wider reach within the vagina. It also allows for greater surface area because the petal like features and flexible silicone material allow the menstrual cup to be compressed within the tampon applicator and expand once the inserted portion is pushed out of the tampon applicator into the vagina. Increased surface area allows for greater cell collection. The menstrual cup with fewer petals (12) allows for a wider rim compared to the menstrual cup with more petals and thinner rim (11). A menstrual cup with tiny bumps (13) or ridges (14) increases surface area. The bumps and ridges can rub the walls of the vagina in a safe and non-irritating manner, letting the device grab cells when it is being removed from the vagina. The surface area and texture let the device collect more cells. A menstrual cup with cilia-like texture all around the cup (15) allows the device to brush cervical cells (and other cells) in the vagina and capture cells on the cilia and in between cilia; again, it also increases surface area and thus there is more surface area to collect cells.

As a further aspect of the invention, it is contemplated that the device can be used to collect cervical and vaginal cells to test for other diseases, such as chlamydia and gonorrhea, and other cancers.

FIG. 4 shows the process for inserting the tampon and using the test tube. The self-collection device is used similarly to a tampon. The intra-vaginal insertion member (3) is inserted into the vagina and the bottom piece is pushed up, pushing out the collection member (9), which is a silicone cup and resembles a mini menstrual cup. When the intra-vaginal insertion member (3) is removed, the inserted portion (9) stays inside the vagina to collect cells. Collection occurs through contact with the vaginal wall and cervix. After a woman wears the inserted portion of the device for a short period of time (preferably less than 20 minutes, but can range from 2 minutes to an hour), the inserted portion is pulled out of the vagina using the attached string (8). The user will drop it into the test tube (4), twist the test tube cap (5) and press the button (10) which will drop down bio-stabilizing liquid.

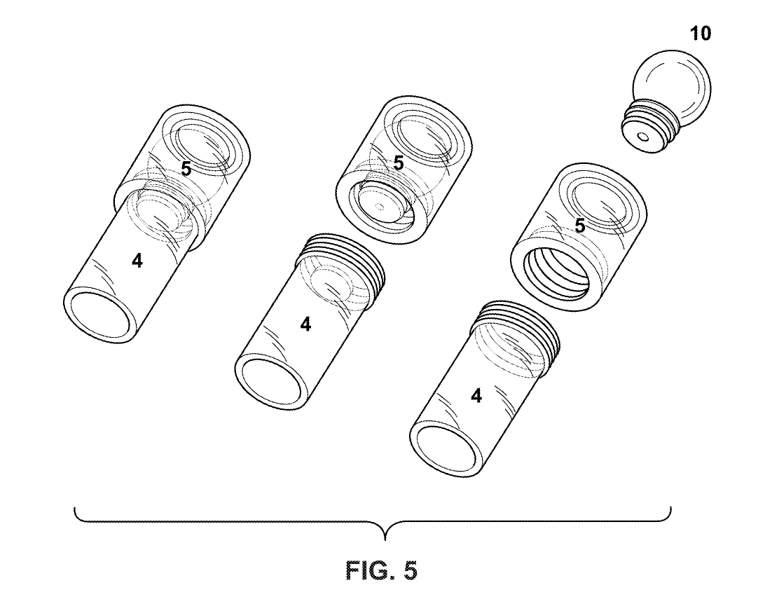

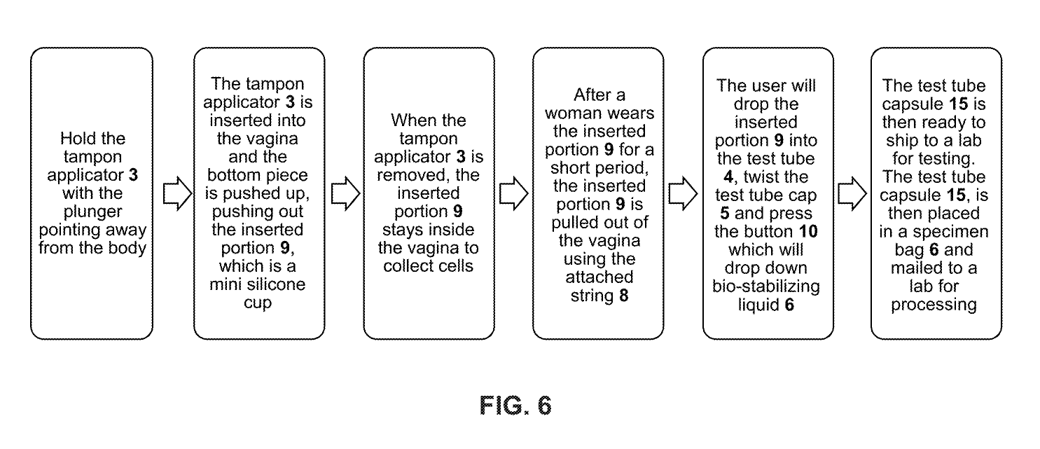

FIG. 5 shows the test tube and test tube cap in more detail. The embodiment of the test tube is provided to illustrate aspects of the invention, but the invention is not limited to the listed embodiment. The button (10) is formed by a flexible pouch built into the test tube cap (5) and the button holds the bio-stabilizing liquid. The test tube cap (5) has an outer thread that screws on to the test tube (4) and an inner thread that screws the pouch into the cap. The top of the cap is opened to expose the top of the pouch, thus resembling a button for the user. The opening of the pouch is located at the bottom of the cap where the cap is screwed onto the test tube, and the opening is sealed by an adhesive. When the user presses the exposed top of the pouch (the button) (10), it puts pressure on the pouch, breaking the seal, opening the pouch, and releasing the bio-stabilizing liquid into the test tube. This bio-stabilizing liquid is methanol-based so it preserves the sample and DNA at room temperature and deactivates any live pathogens to allow for safe and stable shipping. The test tube capsule (16), FIG. 4, is then ready to ship to a lab for testing. The test tube capsule (16), FIG. 4, is then placed in a specimen bag (7), FIG. 1, based on local shipping guidelines, and mailed to a lab for processing and analysis of the sample contained therein.

The tube is shaped and sized such that it can be incorporated into automated lab processes after shipment. Current lab processes use equipment to automatically pipet the bio-stabilizing solution for the testing of cells. The equipment and process to pipette a portion of the sample will remain unchanged.

Another embodiment of the bio-stabilizing liquid releasing mechanism involves a sharp, pin-like object affixed to the underside of the button on top of the lid which is used to puncture the pouch containing the bio-stabilizing liquid. When the user pushes down the button, the pin-like object is driven down into the pouch thus breaking the pouch and releasing the bio-stabilizing liquid into container.

Another embodiment includes a protective cap on top of the button. The cap is intended to prevent mishandling of the button and prevent users from accidentally pressing the button.

Numerous modifications and variations in the practice of the invention are expected to occur to those skilled in the art upon consideration of the foregoing description on the preferred embodiments thereof. Consequently, the only limitations which should be placed upon the scope of the present invention are those that appear in the appended claims.

What is claimed:

1. A self-collection device comprising an intra-vaginal insertion member; a shaped collection member for collecting vaginal and cervical cells and a retrieving member.

2. The device of claim 1 wherein the collection member is a flexible elastomer.

3. The device of claim 2 wherein the flexible elastomer is silicone.

4. The device of claim 1 wherein the collection member has a cupular shape sized to conform to the vagina.

5. The device of claim 4 wherein the collection member comprises protrusions on its exterior.

6. The device of claim 5 wherein the protrusions are circumferential ridges.

7. The device of claim 5 wherein the protrusions are raised protuberances.

8. The device of claim 1 wherein the collection member comprises a plurality of petals expanding from a central core.

9. The device of claim 1 wherein the retrieving member is a string.

10. A kit comprising the self-collection device of claim 1, a container with bio-stabilizing solution and instructions.

11. The kit of claim 10 comprising a tube and cap.

12. A method of collecting vaginal and cervical cells for analysis comprising the steps of disposing inserting the shaped collection member of the self-collection device of claim 1 into the vagina of a subject to be tested,
collecting a biological sample therefrom, and
disposing the collection member in a bio-stabilizing liquid for storage and transport.
